(19) United States
(12) Patent Application Publication
(10) Pub. No.: US 2004/0236339 A1
(43) Pub. Date: Nov. 25, 2004

(54) Long bone reaming apparatus and method

(76) Inventor: John R. Pepper, Cheshire, CT (US)

Correspondence Address: Charles N.J. Ruggiero, ESQ., OHLANDT, GREELEY, RUGGIERO & PERLE, L.L.P., 10th FLOOR, ONE LANDMARK SQUARE, STAMFORD, CT, 06901-2682, US

(21) Appl. No.: 10/869,614

(22) Filed: Jun. 16, 2004

Related U.S. Application Data
(63) Continuation-in-part of application No. 10/369,737, filed on Feb. 21, 2003.
(60) Provisional application No. 60/551,421, filed on Mar. 9, 2004.

(51) Int. Cl. A61B 17/56

(52) U.S. Cl. 606/80

(57) ABSTRACT

A rod is used to guide a cutter through the intramedullary canal of a long bone. Loading and unloading of the cutter is done quickly by having a reduced cross-section near one end of the rod. Loading and unloading of the cutter can also be done quickly by having at least a portion of the rod be flexible so that the ends can be bent. The cutter can have a corresponding slot radially extending from its center. The cutter can be disengaged from a driving shaft without disengaging the driving shaft from the rod, thereby eliminating a difficult realignment process and making the whole cutting process faster. The slot would not interfere with the cutting operation, nor allow the cutter to break free. The reduced section of the rod can be modular and replaceable.

### RELATED APPLICATIONS

This application is related to, and claims priority in, co-pending U.S. Provisional Application Serial No. 60/551,421, filed Mar. 9, 2004, the disclosure of which is incorporated herein by reference. This application is also a continuation-in-part of co-pending U.S. application Ser. No. 10/369,737, filed Feb. 21, 2003, the disclosure of which is incorporated herein by reference.

### BACKGROUND OF THE INVENTION

The treatment of long bone fractures typically involves internal stabilization. The benefits over external stabilization casting traction and plating include, better alignment, less invasive procedure, faster weight bearing, faster recovery, and less blood loss. An intramedullary nail or rod is a cylindrical usually hollow rod inserted in the center of the intramedullary canal or marrow cavity. The rod is usually titanium or stainless steel and is strong enough to support the bone loads during the bones healing process. The bones that are fixed in this manner are the femur, tibia, humerus and radius an ulna. Nails are typically generally circular in cross section or have a shape nearly circular.

One step in the surgical technique is the preparation of the canal. It varies in shape depending upon the position along the bone axis. The center of the bone is called the isthmus, which is a narrowing of the canal. This is especially true in the femur where in order to carry the body weight, the bone gets quite thick in its center.

A nail must be sized to carry the body weight. In order to get present day nails large enough to carry these loads, typically one and one-half to three times body weight, the rod must be larger than the canal. What is presently done is enlarging the canal. This is done is the following steps. A guide rod is inserted in the canal over it entire length. It is usually about two to about four millimeters in diameter and is about 250 to about 1000 millimeters long. It serves several purposes; first it is used to align the fragments of bone. A surgeon will use the guide rod to thread the segments through their canals. Secondly the guide rod is used to guide cutters through the canal to enlarge it to accept the nail.

The reaming process has several components. A drill with a hole through its driving axis is used to supply power. Bone is difficult to cut, especially in young males, who are frequently the fracture patient. The drill is either pneumatic or battery powered. It can impart a high torque on all driving components in order to ream the bone. As the canal of the bone is curved, a flexible shaft is used to couple the drill and the reamer. The flexible shaft is usually about 450 millimeters long and about eight to about twelve millimeters in diameter. It is cannulated, and has a hole along its length slightly larger than the guide rod. It has a connection means on one end for the drill and on the other end for connection with the reamer head (cutters). The connection to the cutter can be a radial or side-loading dovetail. It can also be an axial loading quick connect that uses the guide rod as a lock or locking means.

The reaming set has a set of cutters that increase in diameter in increments of one half to one millimeter. A set of reamer heads for the femur may have reamer heads from nine to fourteen millimeters. The reamers are typical less than three centimeters in length, as longer cutter could not follow the curve of the bone. The reaming is done to one half to one millimeter over the selected nail size so it is easy to insert.

The reaming is done by attaching a reamer head to the flex shaft and then threading these two parts over the end of the guide rod. They are advanced to bone, cut the bone and are withdrawn. The cutter is pulled back off the guide rod and then disconnected from the flexible shaft by moving the cutter radially. The next size reamer head is connected to the flexible shaft radially, and then the assembly is axially threaded onto the guide rod and the process is repeated until the desired cavity size is prepared.

It is common for six to ten reaming steps to be needed to make the canal of sufficient size to accept a nail. This is a slow and tedious part of the surgery. The difficulty arises in that the guide rod is very long and is only slightly more rigid than a coat hanger. The flexible shaft is also pretty flimsy and almost as long. The guide rod's long length outside the body make it a hard target to hit as a three millimeter rod must be axially aligned with a three millimeter nominal sized hole in the flexible shaft with only about a quarter millimeter of tolerance. Two long flexible parts must be perfectly axially aligned in order to be threaded.

These steps show how it is done. The surgeon must hold the dovetail style cutter to the flexible shaft while trying to do the threading process. At the same time, the drill must be supported. This process usually requires at least three hands: one to hold the drill, one to hold the flex shaft reamer connection and one to hold the guide rod steady so they can be axially aligned. As the surgeon's gloves at this time are wet with blood and fat from the canal, they are very slick and only makes this more difficult. Some surgeons that are very skilled can hold the drill with one hand, and use the other to hold the reamer to the shaft, and capture the bouncing guide rod, then align it all together with one hand. Few indeed are those who have this type of dexterity.

The axially loading flex shaft/cutter connections do load these two parts together faster and may hold them together on their own, however they still require three hands to thread the cutter over the guide rod. The most difficult part is threading the cutters on the guide rod, and it must be done many times.

If the reamer head is dropped from the hand onto the floor during this process, it must be sterilized delaying the process even further. Clearly, reaming is a frustrating part of the long bone fracture fixation procedure, and it is no wonder it is left to residents and those in their medical training to do this tedious task.

Another drawback of the contemporary guide rods is that they are constructed of non-resilient or non-flexible material. This provides additional difficulty in manipulating the reamer heads onto the guide rod such as requiring the drill and drill head to be lifted high into the air to perform the exchange.

Accordingly, there is a need for a reaming device and related apparatus that addresses these drawbacks.

### BRIEF SUMMARY OF THE INVENTION

It is an object of the present invention to provide a device that allows changing of reamer heads without removal of the flexible shaft from the guide rod.

It is a further object of the present invention to provide such a device that facilitates the procedure for exchanging cutters during the bone reaming process.

These and other objects and advantages of the present invention are achieved by providing a bone reaming device for reaming a bone canal, which device has a rod and a cutter. The rod has a first end, a second end and is sized and shaped to fit in the bone canal. At least a portion of the rod is flexible thereby allowing the first and second ends to be bent toward each other. The cutter is removably connectable to the rod. The rod guides the cutter in the bone canal.

In another embodiment, there is also provided a guide rod for use with a cutter for reaming a bone canal. The cutter is removably connectable to the guide rod. The guide rod has a body having a first end, a second end and is sized and shaped to fit in the bone canal. The body has at least a portion thereof that is flexible thereby allowing the first and second ends to be bent toward each other. The guide rod guides the cutter in the bone canal.

In another embodiment, there is also provided a guide rod operably connectable with a cutter for creating a hole in a bone canal. The guide rod has a first end, a second end, a flexible portion and a plurality of cross-sectional areas. The guide rod is sized and shaped to fit in the bone canal. At least a first cross-sectional area of the plurality of cross-sectional areas allows the cutter to be removed from the guide rod. At least a second cross-sectional area of the plurality of cross-sectional areas prevents the cutter from being removed from the guide rod. The flexible portion allows the first and second ends to be bent toward each other.

There is also provided a method of reaming a bone canal which includes, but is not limited to, providing a guide rod having a first end, a second end and a flexible portion; providing a cutter that is removably connectable to the guide rod; bending the guide rod at the flexible portion thereby moving the first and second ends toward each other; loading the cutter on the guide rod; and advancing the cutter along the guide rod into the bone canal.

There is also provided a method of reaming a bone canal that includes the steps of, but is not limited to, providing a guide rod having a flexible portion and a longitudinal axis when the flexible portion is unbiased; providing a cutter that is removably connectable to the guide rod; loading the cutter on the guide rod; and advancing the cutter along the guide rod into the bone canal. The loading of the guide rod is done when the guide rod is non-coincidental to the longitudinal axis.

There is also provided a cutter for creating a hole in a bone canal. The cutter has a central bore having a diameter and a radial slot with slot walls. The radial slot is in communication with the central bore. The radial slot has a width that is smaller than the diameter of the central bore.

At least a portion of the rod that is flexible can be made from a super elastic alloy. The second end of the rod may have an enlarged member that prevents the cutter from sliding off the second end. The cutter can have a central bore and a radial slot. The central bore can have a diameter and the radial slot can have a width. The radial slot may be in communication with the central bore. The width of the radial slot can be smaller than the diameter of the central bore.

The rod can have a plurality of cross-sectional areas. At least a first cross-sectional area of the plurality of cross-sectional areas allows the cutter to be removed from the rod and at least a second cross-sectional area of the plurality of cross-sectional areas can prevent the cutter from being removed from the rod. At least one of the plurality of cross-sectional areas may be circular. At least a portion of the first cross-sectional area may be flexible. At least a portion of the second cross-sectional area may be flexible.

The device may also have a support member operably connected between the first and second cross-sectional areas. The support member may be a hollow tube having a third cross-sectional area that is greater than the first cross-sectional area and less than the second cross-sectional area. The support member may have a tapered end. At least a portion of the support member may be made from a super elastic alloy.

The slot walls may be parallel to each other in a direction toward the central bore. The slot walls may converge toward each other in a direction toward the central bore. The slot walls may have first and second portions, where the first portions are parallel to each other in a direction toward the central bore and the second portions converge toward each other in the direction toward the central bore. At least a portion of the cutter may have a coating. The coating can be titanium oxide, chrome, titanium aluminum oxide, or any combinations thereof. At least a portion of the cutter may be treated with a low-friction coating.

The present invention is intended to alleviate the drawbacks of the conventional axially loaded intramedullary reamer used in long bone fixation surgery. An object of this invention is to provide a radially loading reamer that does not necessitate separation of the reamer shaft and the guide rod.

To accomplish the above recited object, the present invention has a long slender rod to fit the intramedullary canal of a long bone with a plurality of cross sectional areas used to guide reamers (cutters) within the intramedullary canal of the bone. The cutters are conventional reaming heads with the addition of a radial slot extending from the central bore. The preferred embodiment has two main shaft cross sections, and both sections are circular. The guide rod can be constructed from one component. The advantage to one component is that is preassembled, however the small diameter shaft can bend during manipulation prior to reaming. Straightening a bent rod intra-operatively can be difficult.

Alternatively, the rod may have more than one segment. One component looks externally like a conventional guide rod with an engagement means on the end opposite the ball end. The engagement means could be contained within an internal cavity, a thread. The second component is a smaller cross section rod, or loading section. The preferred embodiment would be a round section.

The small section could have an engagement means on it. It can be a friction fit into a smooth bore. The preferred embodiment is a thread. The two components could line up axially and lock together. They would then function like the unitary component device. The small component could be added after the fracture manipulation is complete lessening the chance for a bent small section. The small section rod could be a commonly used orthopaedic wire, or Kirschner wire (K wire), used for a multitude of procedures.

The length of the small section should be slightly longer than the cutter. It could be much longer than the cutter, as K wires can be over ten centimeters long. The preferred embodiment for ease of loading, flexible shaft retention would be approximately five centimeters. That typically would allow a few centimeters of small shaft to extend beyond the cutter to hold the flexible shaft in place.

The locking means between each rod segment holds the small section on while the flexible reamer is being moved back and forth. There is some friction between the guide rod and inner portion of the flexible reamer. Axial resistance to the motion of the small segment relative to the large segment could be done with a threaded connection. The reamers tend to run in one direction only, so a standard right hand thread would tend to self tighten during operation. Typical sizes of the rod main portion would be from two and four millimeters in diameter, and the smaller cross section is between one to two millimeters in diameter. The smaller section would typical have a size that is fifty percent of the larger section.

The small cross section can be made by removal of material from a conventional guide rod. This can be done in one or more planes, so the cross section can form a polygon. These cuts can be adjacent to the end of the guide rod, or they can be located a short distance from the end. The later embodiment allows a full section of the guide rod to center the flexible shaft and its dovetail (or equivalent locking means) over the rod to further speed reamer loading. This method is somewhat more difficult to manufacture, as working with a long flexible rod is difficult.

The loading section could have both small and large sections, allowing radially loading while maintaining the centering of a full section on the flexible shaft bearing surface, at the same time, keeping the economy of a constant section main guide rod. This embodiment of the loading section can also be replaceable to reduce bending risk.

Another loading section has two diameter sections equal to the main guide rod flanking a smaller loading section. This allows the advanced centering, radially loading of the previous embodiments, and provides an abutment surface to stop the thread engagement, stiffening the junction between the main guide rod and the loader section. The loading section could have a tapered approach to facilitate loading of the reamer shaft initially.

The small cross section of the above embodiments is long enough to clear the length of the reamer, approximately three centimeters. In cases where the small section is not backed up with a larger cross section, the shorter section can be extended to maintain the flexible shaft on the rod. An overlap of two centimeters is adequate to keep the flexible shaft in place. The length of the straight short portion could then be about five centimeters.

The main portion of the two piece embodiment would be from about 250 to about 1000 millimeters long. This depends on the bone that is being reamed. Generally, the rod is about twice the length of the canal of the bone. The main portion of the two piece assembly has a stop on the end going into the canal to prevent reamer dissociation. The unitary guide rod has a stop to prevent reamer dissociation also.

The reamer or cylindrical cutter enlarges the intramedullary canal by cutting a round hole. This hole will provide means to place an intramedullary rod. The cutters generally are tapered or barrel shape to follow previous cutters, and have a good cutting action. The radial slot is cut from the central bore to the outer edge. It is located to minimize the disturbance to the cutting edges of the flutes. Flutes that must be divided are done so such that there are no weak sections or unintended sharp edges. The slot is slightly wider than the small section of the rod. The reamer head can then slide on and off of the rod. When the reamer is advanced onto the main portion of the rod, it spins freely and can not move radially because the slot is smaller than the guide rod at that portion. At this point it functions like a typical reamer.

When the reamer is to be exchanged, the flexible drive shaft draws it back out of the canal and up the rod so the reamer head is over the smaller section. The cutter is slid off the guide rod along a radial path. When the loading section is a constant diameter (K wire) the flex shaft is held in place and provides some movement between the rod and flexible shaft connector. With the multiple diameter loading section, the larger upper section perfectly centers the flexible shaft so that no alignment is needed. The round cross section of the smaller section does not require special alignment either. The only alignment necessary is that of the reamer dovetail to the flexible shaft, which is as it is required on present reaming systems. In the embodiment of a guide rod with the polygon shaped reduced section, the reamer engagement must be aligned with the polygon before the reamer can be loaded. With all of the embodiments, once the smaller reamer is removed, the next sized reamer is placed over the small section, locked with the dovetail and advanced into the canal.

Another embodiment is for the transitions in guide rod diameters to have tapers to make it easier for a reamer to go from one to another without getting caught. This can be adapted to all previous embodiments. The extra section for the two pieces can have driving means on one end to lock the threads in place and to remove it if need be. These can be a screw driver slot, external or internal polygon shape or a surface geometry such as a knurl. In another embodiment of the loading rod, the tip adjacent to the thread has a diameter to facilitate centering within the thread, making the connection faster.

### BRIEF DESCRIPTION OF THE DRAWINGS

FIG. 1 is a perspective view of the prior art showing a long bone (femur), drill, flexible reamer shaft (shortened and simplified) guide rod (shortened), and cylindrical cutter, just prior to reaming the bone;

FIG. 2 is a perspective view of the prior art showing the reamer assembly disengaged from the guide rod in preparation for reamer exchange;

FIG. 3A is a perspective view of the prior art, showing the radial engagement of the reamer and dovetail reamer connection, off the guide rod;

FIG. 3B is a close up perspective view of the prior art, showing the radial engagement of the reamer and dovetail reamer connection, off the guide rod;

FIG. 4 is a perspective view of the prior art, a guide rod with end stop, straight, shown shortened for clarity;

FIG. 5A is a perspective view of the prior art, a ten mm diameter reamer head with the helical cutting teeth omitted for clarity;

FIG. 5B is a perspective view of the prior art, a twelve mm diameter reamer head with the helical cutting teeth omitted for clarity;

FIG. 5C is a perspective view of the prior art, a fourteen mm diameter reamer head with the helical cutting teeth omitted for clarity;

FIG. 6 is a perspective view of an embodiment, the one piece, multiple cross section shaft of guide rod, with the larger cross section portion of the shaft shortened for clarity of the present invention;

FIG. 7A is a perspective view of another embodiment of the present invention, a multi-piece, multiple cross section shaft guide rod, assembled, with the larger cross section portion of the shaft shortened for clarity;

FIG. 7B is a plan view of a cross section of the multiple-piece rod, with the larger cross portion of the section shaft shortened for clarity;

FIG. 7C is a detail of the plan view of threaded connection of the multiple-piece rod;

FIG. 8A is a perspective view of an alternate embodiment of the guide rod with parallel cuts, cut away from end, with the larger cross section portion of the shaft shortened for clarity; (ball end is omitted);

FIG. 8B is a plan view of the alternate embodiment of the guide rod of FIG. 8A with parallel cuts, cut away from end;

FIG. 9A is a perspective view of an alternate embodiment of guide rod with a polygon cross section, cut away from end;

FIG. 9B is a perspective view of the alternate embodiment of the guide rod of FIG. 9A with a polygon cross section, cut away from end;

FIG. 10 is a perspective view of an embodiment showing the positioning of the cutter, flexible shaft, and guide rod components prior to radial loading; The large section of the guide rod is shown shortened for drawing clarity;

FIGS. 11A-F are perspective views of an embodiment showing progressive radial engagement of the cutter guided by the small section and the connector on the shaft;

FIGS. 12A-C are perspective views of the components in FIG. 10 to illustrate the advancement of cutter and flexible shaft from the small cross section to the large cross section of the rod;

FIGS. 13A-C are perspective views of the components in FIG. 10 to illustrate the advancement of cutter and flexible shaft from the small cross section to the large cross section of the rod rotated to show the junction position;

FIGS. 14A-B are perspective views of the embodiment of FIG. 10 to show the radial slot guiding the small cross section;

FIGS. 15A-B are perspective views of the embodiment of FIG. 8A with the cutter and flexible shaft in alignment for radial engagement;

FIG. 16A is a plan view of the loading of cutter relative to intramedullary canal, guide rod assembly and femur;

FIG. 16B is a perspective view of the loading of cutter relative to intramedullary canal, guide rod assembly and femur;

FIG. 17A is a perspective view of the cutter on the guide rod, attached to the flexible shaft, ready to ream the canal;

FIG. 17B is a detailed view of the cutter of FIG. 17A;

FIG. 18A is a perspective view of the prior art cutter with cutting flutes;

FIG. 18B is a perspective view of a cutter of the present invention with cutting flutes and the radial slot;

FIG. 19A is a perspective view of small cross section rod with constant cross section;

FIG. 19B is a perspective detail view of the embodiment of FIG. 19A showing the locking thread and the leading alignment boss;

FIG. 20 is a perspective view of the main guide rod of large cross section with a threaded recess for the second, small section rod component;

FIG. 21A is a perspective view of a two piece embodiment of the present invention with dual diameters with a flexible shaft alignment boss on the small section component shown assembled;

FIG. 21B is a plan view of the two piece embodiment of the present invention with dual diameters with a flexible shaft alignment boss;

FIG. 22 is a perspective view of the cutter on the two piece embodiment with a flexible shaft alignment boss;

FIG. 23 is a perspective view of the two piece embodiment with a flexible shaft alignment boss and flange adjacent to the locking thread;

FIG. 24 is a plan view of the small section with a driving mechanism, and the flange adjacent to the locking thread;

FIG. 25 is a perspective view of the embodiment shown in FIG. 24;

FIG. 26 is a perspective view of a one piece guide rod with integral small section and upper large section for alignment;

FIG. 27 is a plan view of an alternative embodiment of the guide rod of the present invention;

FIG. 28 is a perspective view of a cross-section of the guide rod with a strain relieving tube;

FIG. 29 is a perspective view of an alternate embodiment of the cutter of the present invention;

FIG. 30 is a top view of the cutter of FIG. 29;

FIG. 31 is a top view of an alternative cutter of the present invention;

FIG. 32 is a top view of another alternative cutter of the present invention;

FIG. 33 is a top view of yet another alternative cutter of the present invention; and

FIG. 34 is a top view of still yet another alternative cutter of the present invention.

### REFERENCE NUMBERS IN THE DRAWINGS

10 femur

20 drill

30 flexible reamer shaft

40 cutter

50 guide rod,

60 axial separation

70 dovetail

75 internal cannula

80 relief

90 dovetail cavity

100 relief channel

110 cannulation

110 cutter cannula

120 stop or ball end

130 two cross sectioned shaft guide rod with ball end.

140 small cross section portion

150 junction

160 large cross section portion

170 small section

180 large section

190 threaded portion

200 threaded recess

210 rectangular

220 square

230 cutter

240 radial slot opening

250 bone's canal

260 cutting flutes

270 recesses.

280 alignment section

290 integral flexible shaft alignment section

300 large sections.

310 driving means

500 guide rod

501 longitudinal axis of guide rod body

600 support member

610 outer edge

620 tapered edge

2300 cutter

2310 cutting flute

2320 leading edge

2325 trailing edge

2350 slot wall

2400 cutter

2410 cutting flute

2450 slot wall

2500 cutter

2510 cutting flute

2550 slot wall

2600 cutter

2610 cutting flute

2650 slot wall

2655 parallel wall

2660 angled wall

2700 cutter

2710 cutting flute

2750 slot wall

2755 parallel wall

2760 angled wall

### DETAILED DESCRIPTION OF THE INVENTION

For purposes of promoting an understanding of the principles of the present invention, reference will now be made to the embodiments illustrated in the drawings and specific language will be used to describe the same. It will nevertheless be understood that no limitation of the scope of the invention is thereby intended, there being contemplated such alterations and modifications of the illustrated device, and such further applications of the principles of the invention as disclosed herein, as would normally occur to one skilled in the art to which the invention pertains.

FIG. 1 shows the prior art. A long bone, in this case a femur 10, is reamed with a drill 20 coupled to a flexible reamer shaft 30 and a cutter 40. This assembly is slid over a guide rod, 50 into the intramedullary canal. The flexible shaft 30 and the cutter 40 are locked together so they rotate at the same speed. The drill drives these two components into the bone 10, to create a cylindrical cavity for a fracture fixing rod.

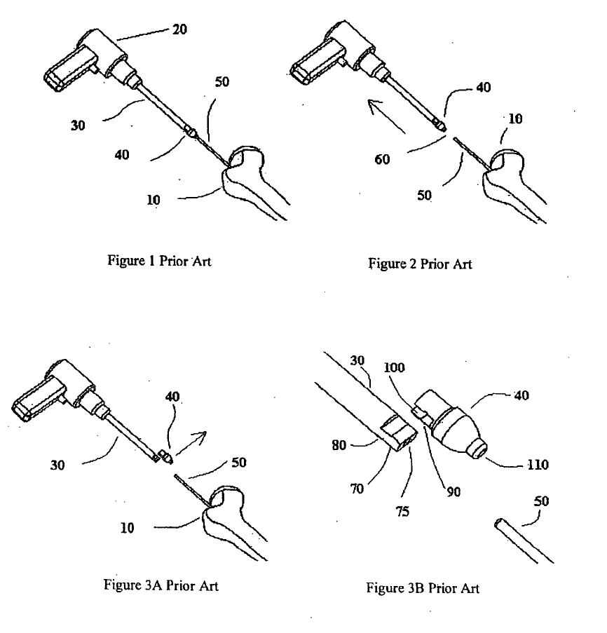

FIG. 2 is of prior art showing the axial separation 60 of the cutter/flexible shaft/drill and the guide rod 50 in preparation for reamer exchange.

FIG. 3A is of prior art showing the radial loading of the cutter 40 onto the flexible shaft 30.

FIG. 3B is of prior art showing details of a typical flexible shaft and reamer connection. The flexible shaft has a dovetail 70 adjacent to a relief 80. The cutter has a corresponding dovetail cavity 90 and smaller relief channel 100. The cuter has a cannulation 110 extending through its length. The cannulation 110 is slightly larger than the guide rod 50. The flexible shaft 30 has an internal cannula 75 that is the same size as the cutter cannula 110 and both of these are slightly larger than the guide rod 50, so everything will easily rotate about the guide rod 50, when powered by the drill.

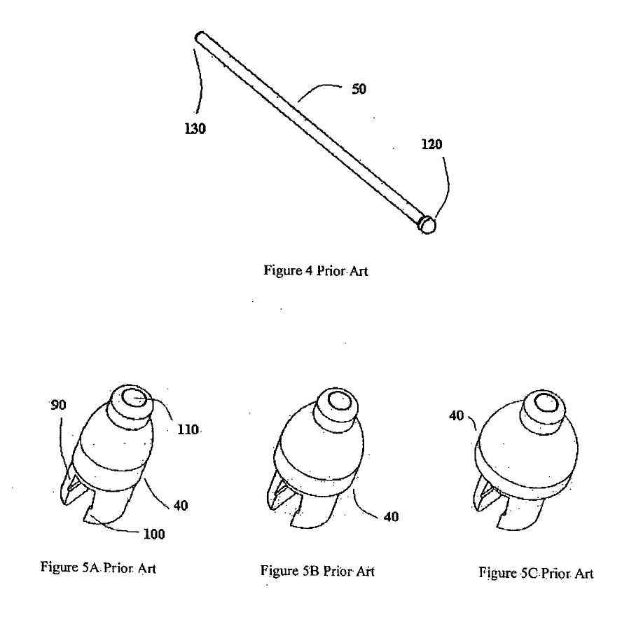

FIG. 4 shows the prior art guide rod 50. It is typically a solid rod with a stop or ball end 120. The ball end 120 is larger than the cutter cannulation 110 and will not allow the cutter to pass. This keeps the cutter from coming off inside the bone. The ball is typical welded or silver soldered onto the rod. The rod can be from 300 to 1000 millimeters long.

FIGS. 5 A,B,C show the prior art. The cutters have identical dovetail cavity and reliefs, but the main diameter increases.

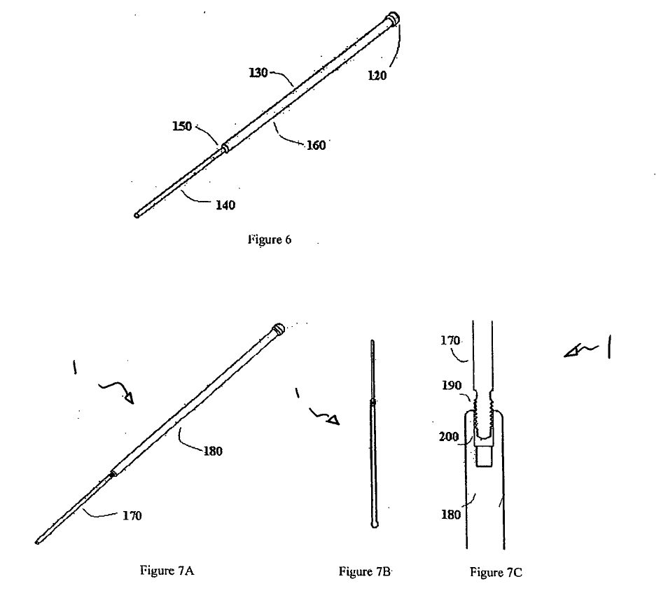

FIG. 6 shows the two cross sectioned shaft of rod 130 with ball end of one embodiment of the present invention. The rod 130 has a large cross section portion 160, a small cross section portion 140 and a ball end 120. The junction 150 of the large cross section portion 160 and small cross section portion 140 is tapered to facilitate cutter transfer. The components line up along their axes. The large cross section 160 and the ball end 120 are similar in shape to the prior art.

FIGS. 7A and B show the inventive guide rod 1 that can be more than one component. The small section rod component 170 can be exclusively on one component, and the large section on another component 180. The two components 170, 180 are joined together to functional as one with a connection.

A threaded connection is shown in FIG. 7C. The small section rod component 170 has a threaded portion on one end 190, mates with a threaded recess 200 in the large section rod component 180.

The cross section of the smaller section rod component 170 is shown as circular.

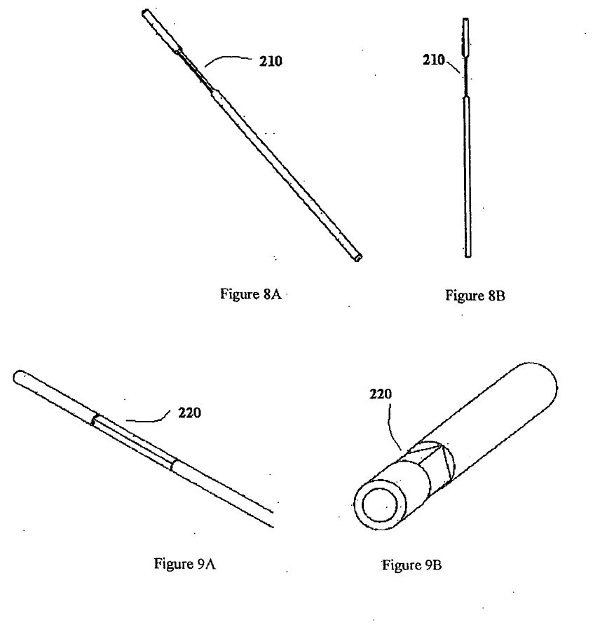

The smaller cross section can be non circular and can be generally rectangular 210 or square 220 as shown in FIGS. 8A-B and FIGS. 9A-B.

FIG. 10 shows a flexible shaft 30 that goes over the guide rod small section 170. The inventive cutter 230 is positioned so the dovetail locking feature 90 is aligned with the mating geometry on the flexible shaft 70 and the inventive radial slot opening 240 is directed toward the small section.

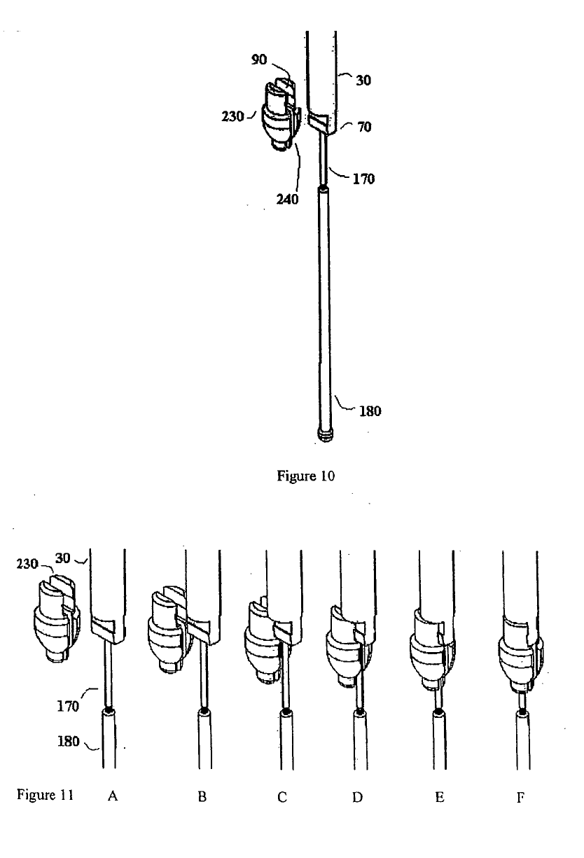

FIGS. 11A-F show the cutter 230 advanced radially toward the center of the guide rod small section 170.

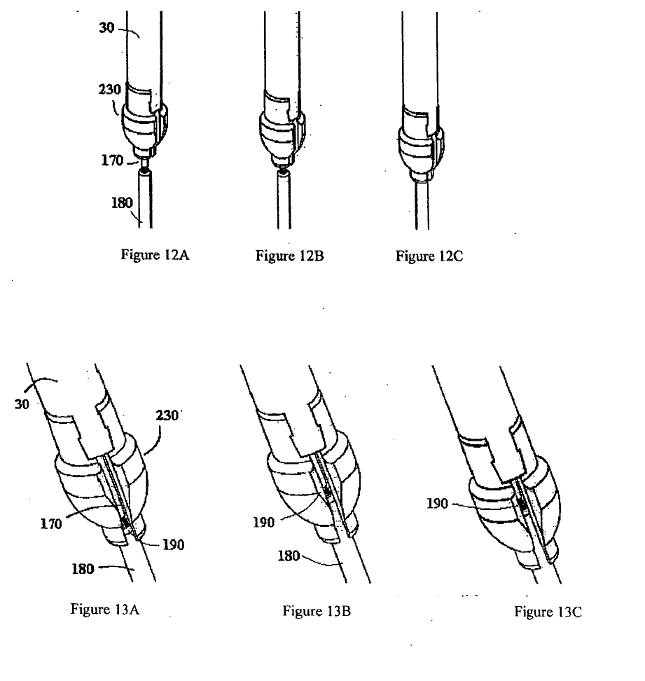

FIG. 12A-C show the cutter 230 and flexible shaft 30 advanced axially down the guide rod 180.

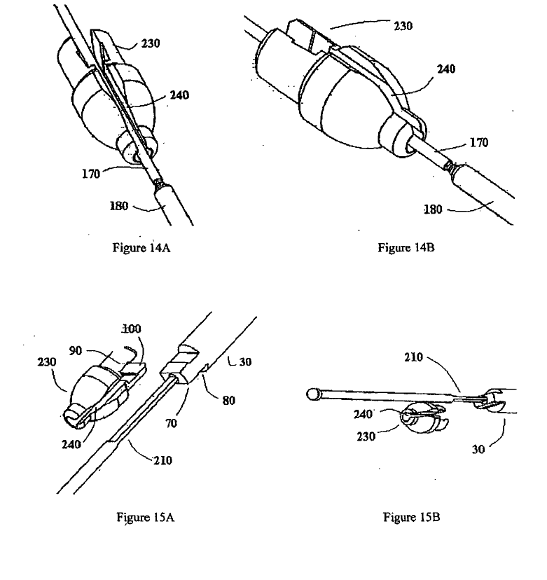

FIG. 13A-C show the smaller section of the guide rod 170 going into the flexible shaft 30 and the inventive cutter cannula or center bore 110 fitting over the guide rod large section 180.

FIGS. 14A and B show the detail of the cutter retention means. The small section 170 can pass through the radial slot 240. The large section 180 can not, and the cutter 230 can freely rotate on the main rod section 180.

FIGS. 15A and B show the non round smaller section guide rod 210 align with the flexible shaft dovetail 80. The geometry on the guide rod 210 must be aligned with the slot 240 in the cutter 230 to advance the cutter radially to the locking position.

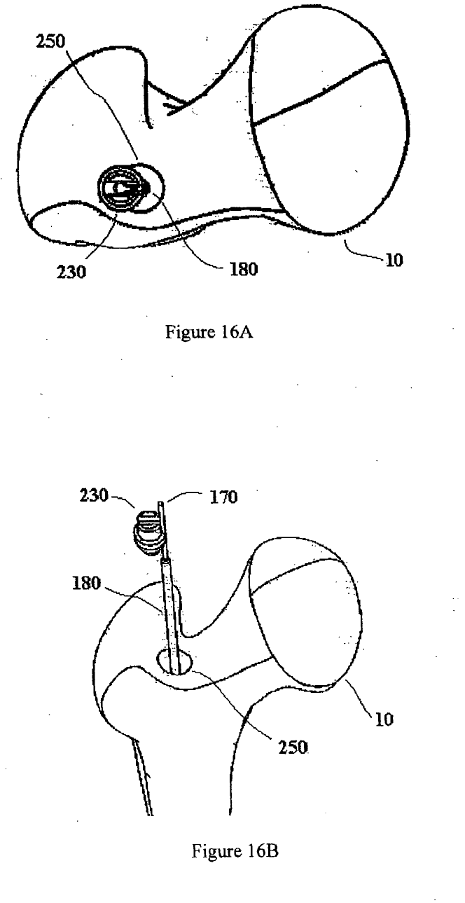

FIGS. 16A and B show that the loading of the cutter 230 is done adjacent to the bone 10. Most of the guide rod large section 180 is in the bone's canal 250 while loading occurs.

FIGS. 17A and B show the cutter 230 with the radial slots 240 positioned ready to go into the intramedullary canal 250. The drill 20 and flexible shaft 30 advance the cutter 230 over the guide rod large section 180 into the canal 250.

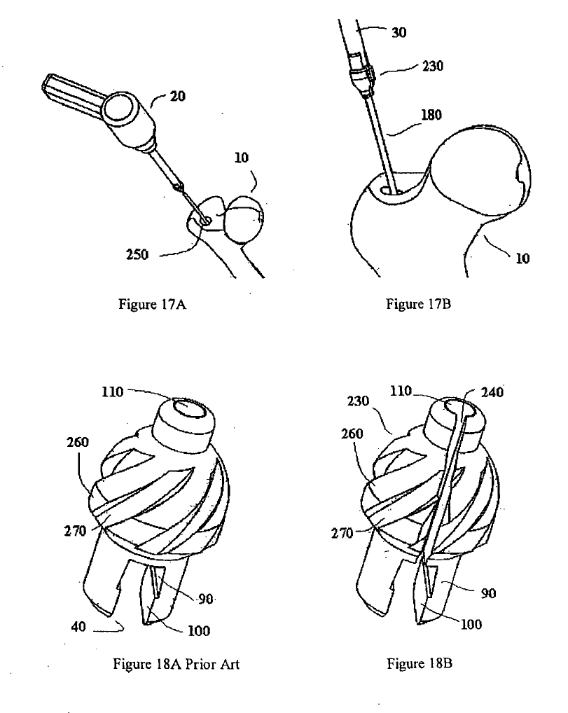

FIG. 18A shows a conventional cutter 40 with cutting flutes 260 and recesses 270 shown.

FIG. 18B shows the inventive cutter 230 with cutting flutes 260, recesses 270 and the inventive radial slot 240 shown. The cannulation 110 of the cutter is the same as the one shown in the prior art cutter 40. The flexible shaft retention means, shown here as a dovetail interlock, 90 and 100, are also the same as the prior art.

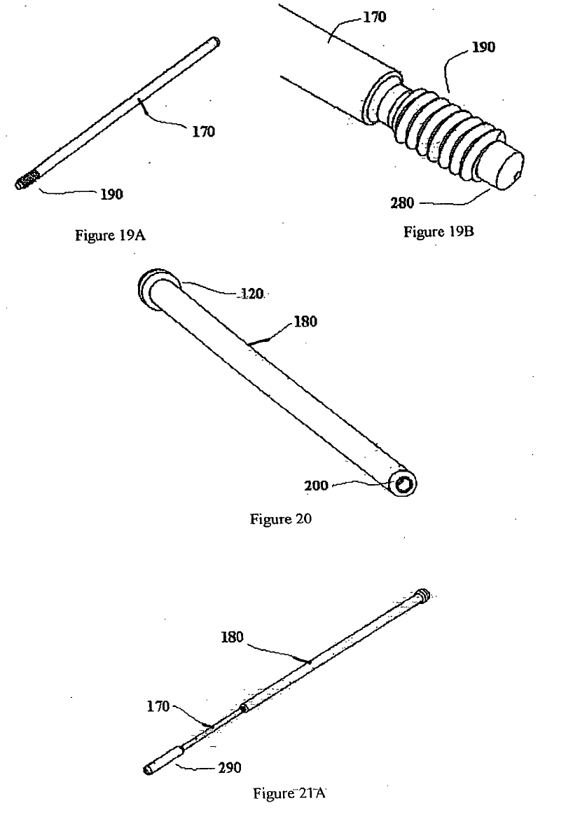

FIG. 19A shows the single cross section small section component 170 of the guide rod assembly. The cross section is circular of maximum stiffness and ease of manufacture.

FIG. 19B shows a detail of the small cross section 170. A thread 190 is used for joining the component of the guide rod, with an alignment section 280 to align and help start the threading process.

FIG. 20 shows the inventive large section guide rod component 180 with a connecter means 200, a threaded hole shown in the non ball end.

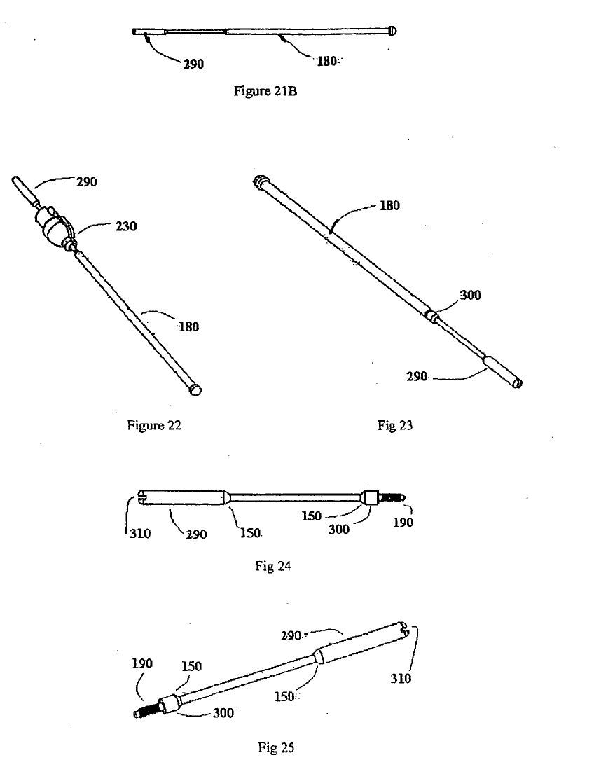

FIGS. 21A and B show the inventive guide rod assembly having a small section segment with an integral flexible shaft alignment section 290. The flexible shaft alignment section has a diameter approximately the same diameter as the main guide rod. The internal cannula of the flexible shaft 290 is a slip fit over the guide rod 180. The flexible shaft alignment section 290 centers the dovetail of the cutter aligned with the dovetail of the flexible shaft so the radial slot does not require its own alignment.

FIG. 22 shows the cutter 230 relative to the small section and the flexible shaft alignment section 290.

FIG. 23 shows an embodiment of the small section guide rod component with two large sections 290 and 300 adjacent each other. The large section 300 adjacent to the threads stabilizes the thread.

FIGS. 24 and 25 shows a driving mechanism or means to engage the small diameter section with a driving tool 310. The driving means 310 is shown as a slot for a screw driver. The tapers 150 are to ease axial travel of the cutter.

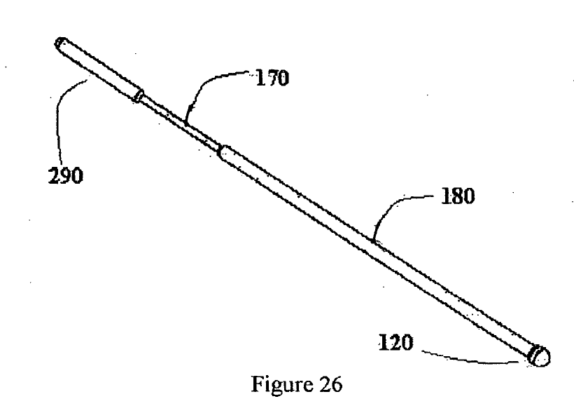

FIG. 26 shows an embodiment of the guide rod with the main section 180, the small section 170 and the alignment section 290 all in one piece.

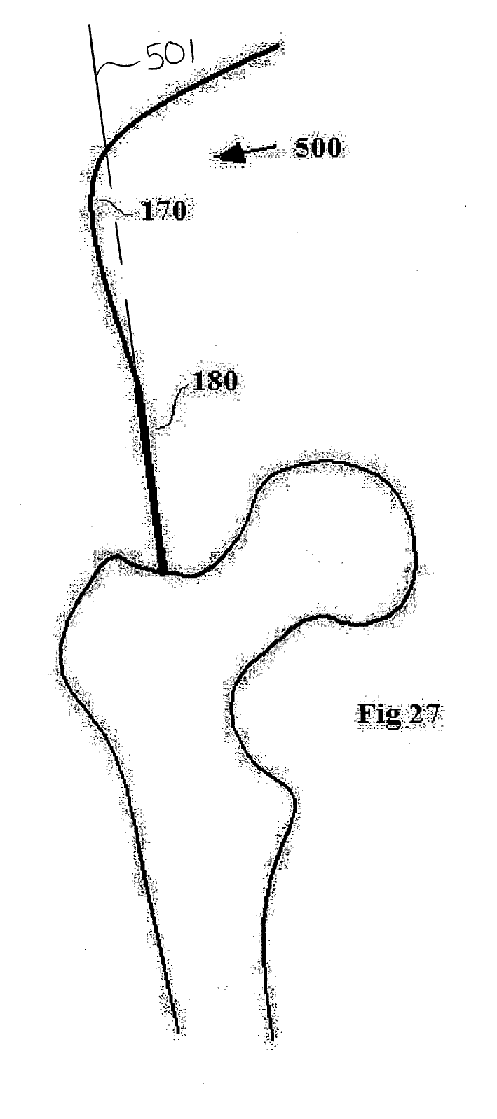

Referring to FIG. 27, another embodiment of the present invention is shown in which at least a portion of the guide rod 500 has a body that is made from a flexible, resilient material to facilitate exchanging out the cutter 230. Preferably, all of small section rod component 170 of guide rod 500 is made from the flexible, resilient material. This allows the large section rod component 180 to be hard and rigid enough to move bone segments, while also allowing the flexible portion to be bent and/or manipulated so that the drill 20 and reamer shaft 30 can be located in an accessible position/orientation when exchanging the cutters 230. This is particularly helpful where longer guide rods 500 are needed and the drill 20 would typically need to be elevated very high to exchange the cutter 230. Reference numeral 501 generally represents the longitudinal axis of the guide rod 500 when in an unbiased or unstressed state, e.g., not bending the ends towards each other.

Alternative positionings of the flexible portion along the guide rod 500 can be used which similarly allow for resilient bending to make the drill more accessible for exchanging the cutters 230, such as, for example, a portion or a plurality of portions of the small section rod component 170, the large section rod component 180, or both, being made flexibly resilient. Also, the entire body of the guide rod 500 or a substantial portion thereof can be made flexible or flexibly resilient.

The portion of the guide rod 500 that is flexibly resilient can be made from a variety of flexible, resilient materials and/or combinations of flexible, resilient materials. Preferably, the flexible material is a shape memory alloy and/or super elastic alloy. More preferably, the material is a nickel titanium alloy or a combination of nickel titanium alloys. Most preferably, the material is nitinol.

Due to the load exerted on the transition between the rigid and flexibly resilient portions of guide rod 500, a taper along the guide rod can be used in the transition area so as to relieve the stress. Also, a tube or other rigid support member could be placed over, around, or be operably connected to the flexible portion of guide rod 500 to provide support and relieve the stress.

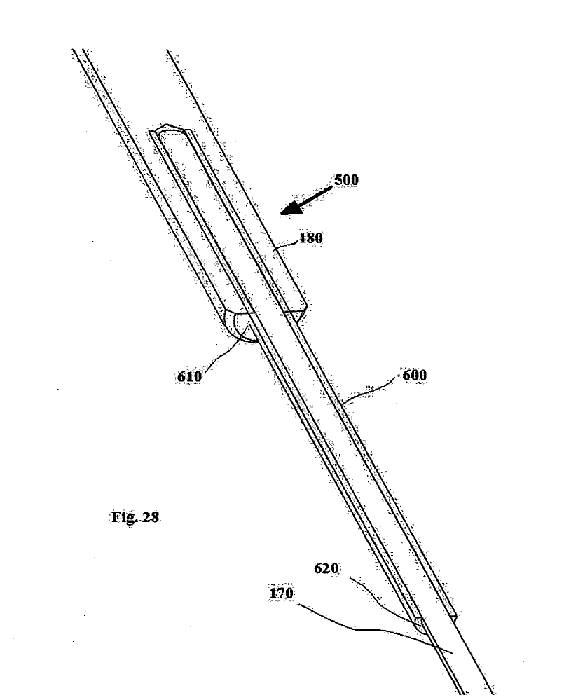

Referring to FIG. 28, a stress or strain relieving hollow tube of the present invention is shown and represented by reference numeral 600. Tube 600 provides stress or strain relief to lessen the localized stress or strain in the transition area between the flexibly resilient area and the more rigid area, which in this particular embodiment is between the small section 170 and the large section 180. Tube 600 also facilitates the cutters 230 (shown in FIGS. 10-17) traveling along the guide rod 500 and passing over the transition area by reducing the change in diameter between the small section 170 and the large section 180 of the guide rod.

Tube 600 can have various shapes to further facilitate both relieving the stress and strain in the transition area and facilitating the cutters 230 passing over the transition area, such as, for example, a small or gradually increasing outer diameter in proximity to the transition area near the outer edge 610 of the large section 180 so that the cutter does not catch that edge. Tube 600 can also have an angled or tapered edge 620 to facilitate the cutters passing from the small section rod component 170 over the tube. The tube 600 can additionally be a plurality of tubes, with the same or different shapes and/or dimensions, to further reduce the stress or strain in the transition area and further facilitate the cutters 230 passing over the transition area.

Preferably, tube 600 is made from a flexible, resilient material or combination of flexible, resilient materials. More preferably, the flexible material is a shape memory alloy and/or super elastic alloy. Even more preferably, the material is a nickel titanium alloy or a combination of nickel titanium alloys. Most preferably, the material is nitinol.

Where the entire small section rod component 170 is made of the flexible resilient material, such as, for example, nitinol, it can be crimped into place in the end of the rigid larger section rod component 180 through a hole disposed through the end of the larger section. Where tube 600 or another support member is used in conjunction with the flexible portion of the guide rod 500, the flexible portion and the tube can be crimped or swaged together, such as, for example, in a hole formed in the end of the rigid portion of the guide rod. The distal end of guide rod 500 has an enlarged member (not shown) that prevents the cutters 230 from sliding off.

The use of a flexible portion for a part or all of the guide rod 500 also facilitates shipping and handling of the guide rod. Conventional guide rods that are made of rigid material require very large boxes for shipping, which is avoided by the present invention. Additionally, cleaning and sterilization is facilitated due to the flexibility of the guide rod 500 which can be manipulated into a smaller area, such as, for example, a sterilization autoclave. The guide rod 500 can also have a clip operably connected thereto and preferably connected to the flexible portion to prevent the flexible portion from leaving the sterile field, such as, for example, by affixing the clip to a surgical drape. The clip can also facilitate the packaging of the guide rod 500, such as, for example, allowing for the flexible portion to be clipped to the rigid portion of the guide rod to reduce the overall footprint.

It should also be understood that the present invention contemplates the flexible portion or plurality of portions (or entirety) of guide rod 500 being usable with the various embodiments described herein. Alternatively, the present invention contemplates the flexible portion or plurality of portions, as described herein, being usable with other guide rods so that they are capable of placing the distal end of the guide rod, which is connectable with the drill 20, in a position that makes the drill more accessible for exchanging the cutters 40, such as, for example, guide rods having a uniform diameter.

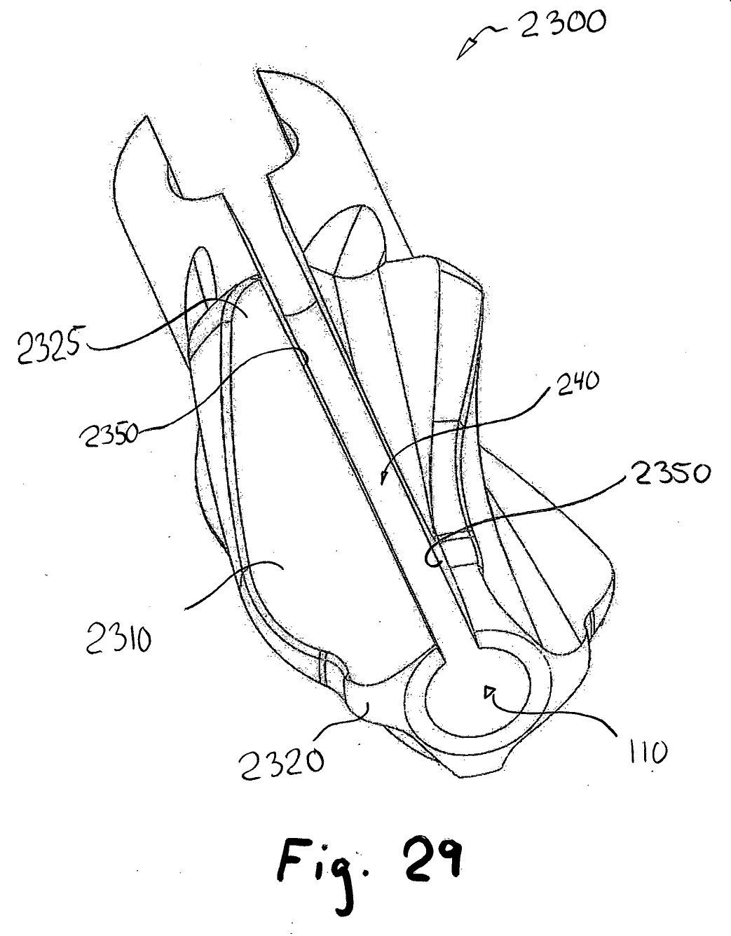

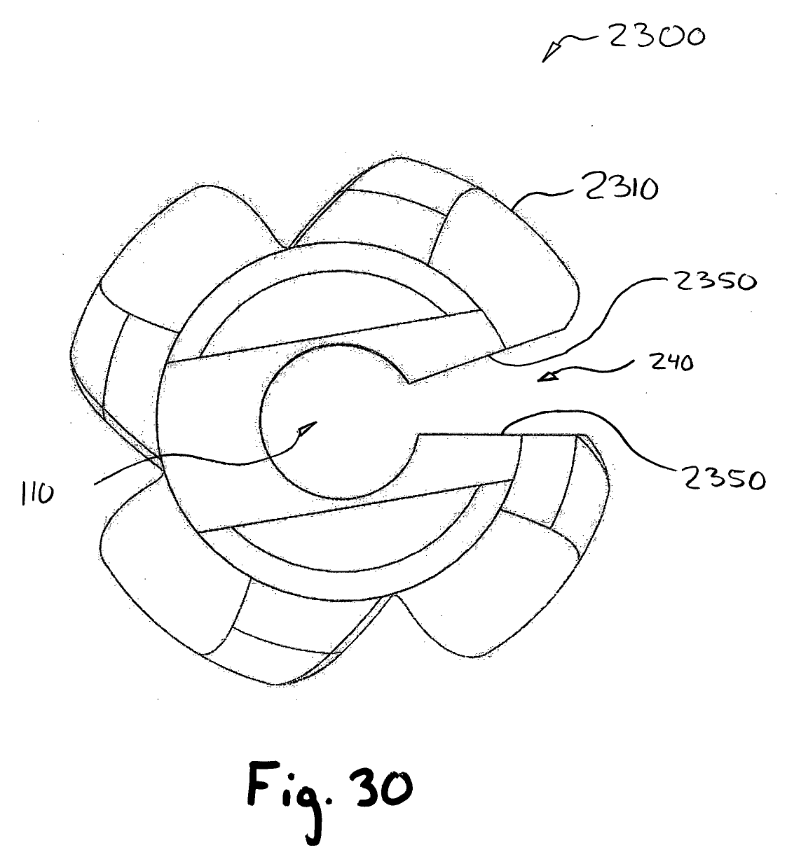

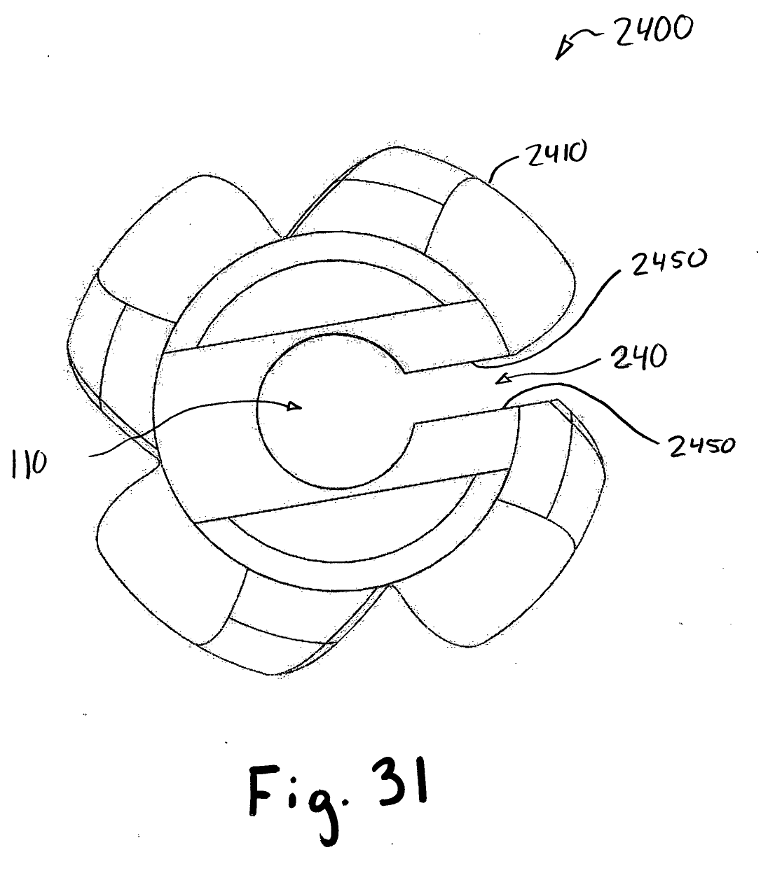

Referring to FIGS. 29 and 30, a cutter of the present invention is shown and generally represented by reference numeral 2300. Cutter 2300, similar to cutter 230 described above, allows for easy loading and unloading of the cutter on the guide rod 130, 500 by way of central bore 110 and slot 240. Cutter 2300 has cutting flutes 2310, which are preferably shaped in a spiral or curved configuration. Cutting flutes 2310 preferably have leading edges 2320 that are chamfered or smoothly shaped to facilitate cutting and manipulation of the cutter 2300. Cutting flutes 2310 are also preferably tapered so that the leading edge 2320 has a smaller width than the trailing edge 2325. The present invention also contemplates the use of other shapes and sizes of flutes 2310 to facilitate cutting and manipulation of the cutters 2300.

Slot 240 in cutter 2300 is defined by slot walls 2350. Cutter 2300 has slot walls 2350 that are non-parallel, symmetric and converging towards each other in the direction of the center bore 110. The converging angle of the slot walls 2350 facilitates the loading of guide rods 130, 500 through the slot 240 and into center bore 110 by providing a larger target. The converging angle of the slot walls 2350 also facilitates ejection of any bone chip that enters the slot 240 since the outer opening of the slot will be wider than the inner opening near the center bore 110.

Referring to FIG. 31, another alternate cutter is shown and generally represented by reference numeral 2400. Cutter 2400 allows for easy loading and unloading of the cutter on the guide rod 130, 500 by way of central bore 110 and slot 240. Cutter 2400 has cutting flutes 2410 with features similar to the ones described above with respect to cutting flutes 2310. Slot 240 in cutter 2400 is defined by slot walls 2450. Slot walls 2450 are parallel. The parallel configuration of the slot walls 2450 reduces the likelihood of bone chips entering the slot 240 as compared to the slot in cutter 2300 since the outer opening of the slot will be the same size as the inner opening near the center bore 110.

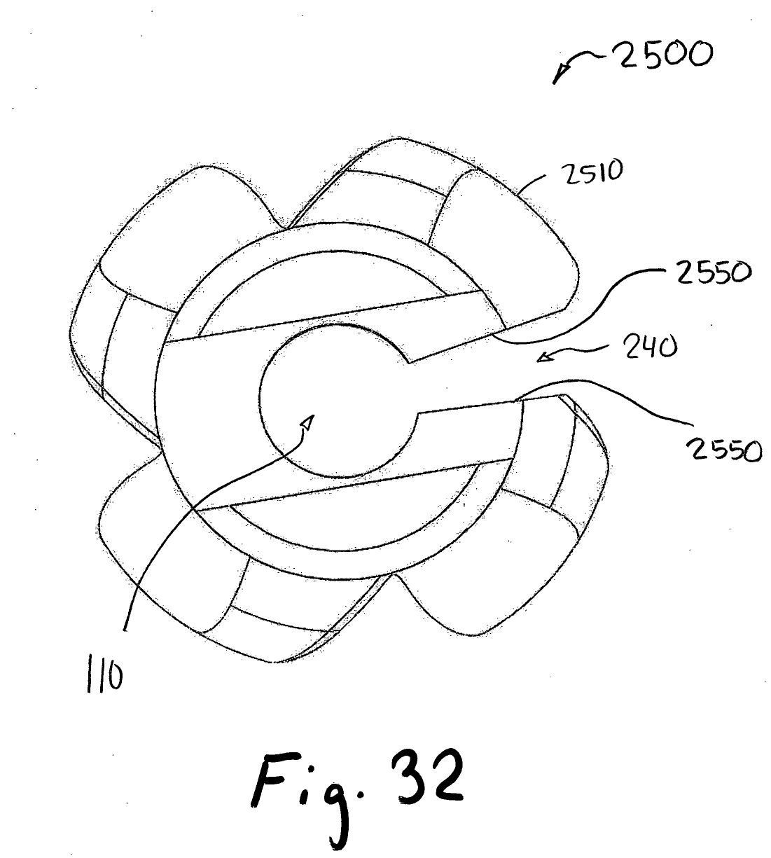

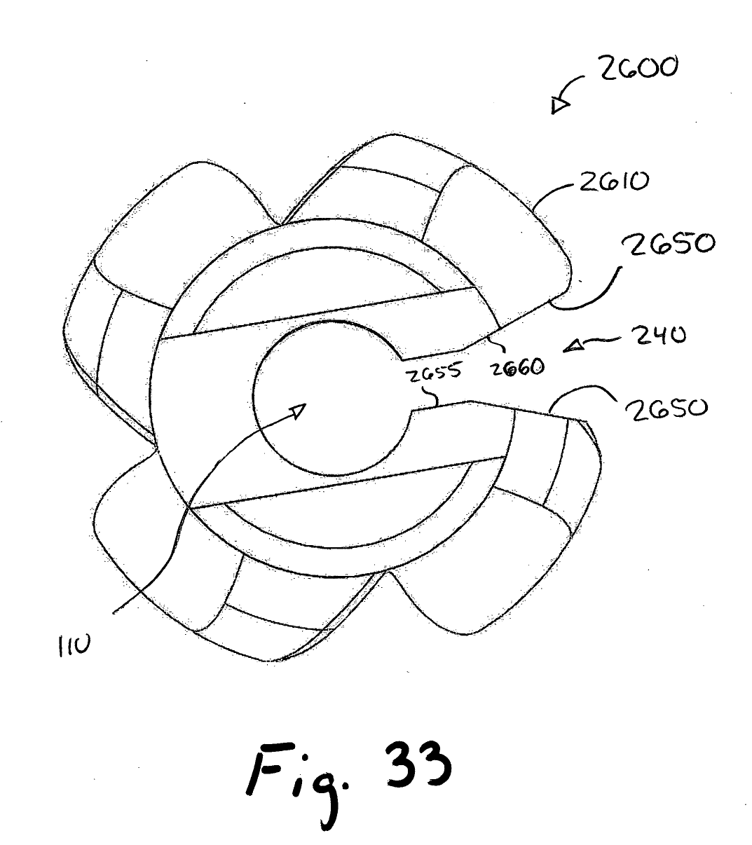

Referring to FIG. 32, another alternate cutter is shown and generally represented by reference numeral 2500. Cutter 2500 allows for easy loading and unloading of the cutter on the guide rod 130, 500 by way of central bore 110 and slot 240. Cutter 2500 has cutting flutes 2510 with features similar to the ones described above with respect to cutting flutes 2310. Slot 240 in cutter 2500 is defined by slot walls 2550. Slot walls 2550 are non-parallel, non-symmetric and converging towards each other in the direction of the center bore 110. The converging angle of the slot walls 2550 facilitates the loading of guide rods 130, 500 through the slot 240 and into center bore 110 by providing a larger target. The converging angle of the slot walls 2550 also facilitates ejection of any bone chip that enters the slot 240 since the outer opening of the slot will be wider than the inner opening near the center bore 110. The non-symmetry of slot walls 2550 reduces the chance of the slot 240 filling with bone chips since the size of the outer opening is being reduced and the reduction of the angle reduces chips being drawn into the slot.

Referring to FIG. 33, another alternate cutter is shown and generally represented by reference numeral 2600. Cutter 2600 allows for easy loading and unloading of the cutter on the guide rod 130, 500 by way of central bore 110 and slot 240. Cutter 2600 has cutting flutes 2610 with features similar to the ones described above with respect to cutting flutes 2310. Slot 240 in cutter 2600 is defined by slot walls 2650. Slot walls 2650 have portions that are parallel 2655 and non-parallel 2660 that converge towards each other in the direction of the center bore 110. The parallel configuration of the wall portion 2655 reduces the likelihood of bone chips entering the center bore 110. The converging angle of the wall portion 2660 facilitates the loading of guide rods 130, 500 through the slot 240 and into center bore 110 by providing a larger target. The converging angle of the slot walls 2660 also facilitates ejection of any bone chip that enters the slot 240 since the outer opening of the slot will be wider than the inner opening near wall portion 2655.

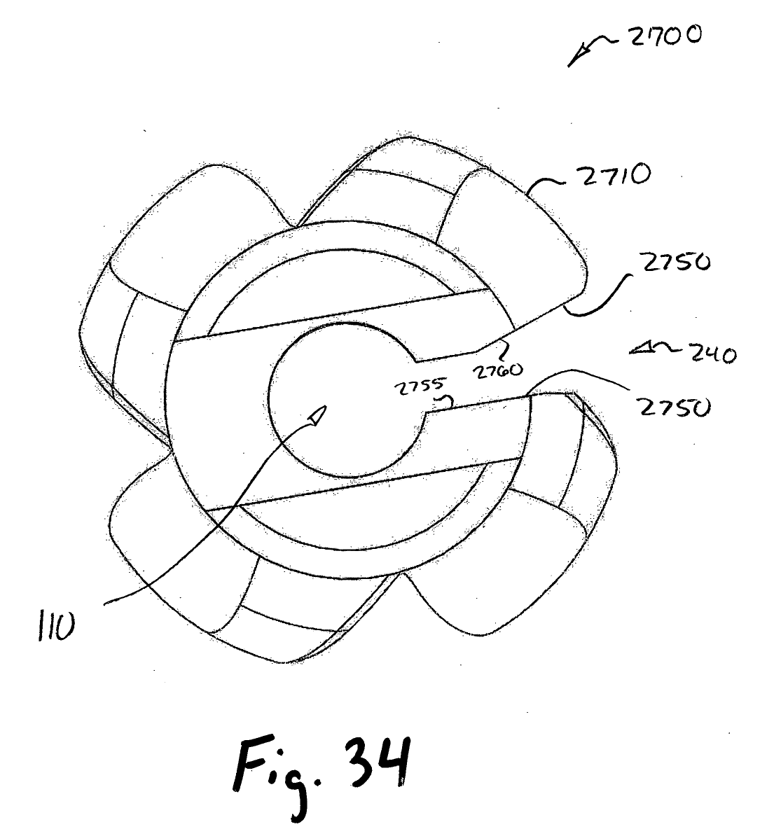

Referring to FIG. 34, another alternate cutter is shown and generally represented by reference numeral 2700. Cutter 2700 allows for easy loading and unloading of the cutter on the guide rod 130, 500 by way of central bore 110 and slot 240. Cutter 2700 has cutting flutes 2710 with features similar to the ones described above with respect to cutting flutes 2310. Slot 240 in cutter 2700 is defined by slot walls 2750. Slot walls 2750 have portions that are parallel 2755, as well as non-symmetrical, non-parallel wall portions 2760 that converge towards each other in the direction of the center bore 110. The parallel configuration of the wall portion 2755 reduces the likelihood of bone chips entering the center bore 110. The converging angle of the wall portion 2760 facilitates the loading of guide rods 130, 500 through the slot 240 and into center bore 110 by providing a larger target. The converging angle of the slot walls 2760 also facilitates ejection of any bone chip that enters the slot 240 since the outer opening of the slot will be wider than the inner opening near wall portion 2755. The non-symmetry of slot walls 2750 reduces the chance of the slot 240 filling with bone chips since the size of the outer opening is being reduced and the reduction of the angle reduces chips being drawn into the slot.

The present invention also contemplates the use of other configurations for the cutters described above, such as, for example, disposing the parallel wall portion of the slot walls closest to the leading edge of the cutter in order to further reduce the likelihood of bone chips entering the slot 240. Any of the cutters described above could also be coated for improved performance, such as, for example, with a hardener. Such coatings include, but are not limited to, titanium oxide, chrome and/or titanium aluminum oxide. The present invention contemplates the use of a marker or indicator to provide visual indication of the slot 240, such as, for example, a coloring. The area in proximity to slot 240 can also be coated with a low-friction substance to facilitate loading the guide rod 130, 500 into slot 240.

The present invention having been thus described with particular reference to the preferred forms thereof, it will be obvious that various changes and modifications may be made therein without departing from the spirit and scope of the present invention as defined in the appended claims.

What is claimed is:

1. A guide rod for use with a cutter for reaming a bone canal, the cutter being removably connectable to the guide rod, the guide rod comprising:
a body having a first end, a second end and being sized and shaped to fit in the bone canal, wherein said body has at least a portion thereof that is flexible thereby allowing said first and second ends to be bent toward each other and wherein the guide rod guides said cutter in the bone canal.

2. The guide rod of claim 1, wherein said at least a portion of said body that is flexible is made from a super elastic alloy.

3. The device of claim 2, wherein said at least a portion of said body that is flexible is substantially all of said body.

4. A bone reaming device for reaming a bone canal comprising:
a rod having a first end, a second end and being sized and shaped to fit in the bone canal, wherein said rod has at least a portion thereof that is flexible thereby allowing said first and second ends to be bent toward each other; and
a cutter removably connectable to said rod, wherein said rod guides said cutter in the bone canal.

5. The device of claim 4, wherein said at least a portion of said rod that is flexible is made from a super elastic alloy.

6. The device of claim 5, wherein said at least a portion of said rod that is flexible is substantially all of said rod.

7. The device of claim 4, wherein said second end of said rod has an enlarged member that prevents said cutter from sliding off said second end.

8. The device of claim 4, wherein said cutter has a central bore and a radial slot, said central bore having a diameter, said radial slot having a width, said radial slot being in communication with said central bore, wherein said width of said radial slot is smaller than said diameter of said central bore.

9. The device of claim 4, wherein said rod has a plurality of cross-sectional areas, wherein at least a first cross-sectional area of said plurality of cross-sectional areas allows said cutter to be removed from said rod, and wherein at least a second cross-sectional area of said plurality of cross-sectional areas prevents said cutter from being removed from said rod.

10. The device of claim 9, wherein at least one of said plurality of cross-sectional areas is circular.

11. The device of claim 9, wherein at least a portion of said at least a first cross-sectional area is flexible.

12. The device of claim 9, wherein at least a portion of said at least a second cross-sectional area is flexible.

13. The device of claim 9, further comprising a support member operably connected between said at least a first cross-sectional area and said at least a second cross-sectional area.

14. The device of claim 13, wherein said support member is a hollow tube having a third cross-sectional area that is greater than said at least a first cross-sectional area and less than said at least a second cross-sectional area.

15. The device of claim 14, wherein said support member has a tapered end.

16. The device of claim 14, wherein at least a portion of said support member is made from a super elastic alloy.

17. A guide rod operably connectable with a cutter for creating a hole in a bone canal, the guide rod comprising:
a first end, a second end, a flexible portion and a plurality of cross-sectional areas, wherein the guide rod is sized and shaped to fit in the bone canal, wherein at least a first cross-sectional area of said plurality of cross-sectional areas allows the cutter to be removed from the guide rod, wherein at least a second cross-sectional area of said plurality of cross-sectional areas prevents the cutter from being removed from the guide rod, and wherein said flexible portion allows said first and second ends to be bent toward each other.

18. The guide rod of claim 17, wherein said flexible portion is made from a super elastic alloy.

19. The guide rod of claim 17, wherein at least one of said plurality of cross-sectional areas is circular.

20. The guide rod of claim 17, wherein at least a portion of said at least a first cross-sectional area is flexible.

21. The guide rod of claim 17, further comprising a support member operably connected between said at least a first cross-sectional area and said at least a second cross-sectional area.

22. The guide rod of claim 21, wherein said support member is a hollow tube having a third cross-sectional area that is greater than said at least a first cross-sectional area and less than said at least a second cross-sectional area.

23. The guide rod of claim 17, wherein said second end has an enlarged member that prevents the cutter from sliding off the guide rod.

24. A cutter for creating a hole in a bone canal, the cutter comprising:
a central bore having a diameter; and
a radial slot having slot walls and being in communication with said central bore, said radial slot having a width, wherein said width of said radial slot is smaller than said diameter of said central bore.

25. The cutter of claim 24, wherein said slot walls are parallel to each other in a direction toward said central bore.

26. The cutter of claim 24, wherein said slot walls converge toward each other in a direction toward said central bore.

27. The cutter of claim 24, wherein said slot walls have first and second portions, wherein said first portions are parallel to each other in a direction toward said central bore, and wherein said second portions converge toward each other in said direction toward said central bore.

28. The cutter of claim 24, wherein at least a portion of the cutter has a coating selected from the group consisting essentially of titanium oxide, chrome, titanium aluminum oxide, and any combinations thereof.

29. The cutter of claim 24, wherein at least a portion of the cutter is treated with a low-friction coating.

30. A method of reaming a bone canal comprising:
providing a guide rod having a first end, a second end and a flexible portion;
providing a cutter that is removably connectable to said guide rod;
bending said guide rod at said flexible portion thereby moving said first and second ends toward each other;
loading said cutter on said guide rod; and
advancing said cutter along said guide rod into the bone canal.

31. A method of reaming a bone canal comprising:
providing a guide rod having a flexible portion and a longitudinal axis when said flexible portion is unbiased;
providing a cutter that is removably connectable to said guide rod;
loading said cutter on said guide rod; and
advancing said cutter along said guide rod into the bone canal,
wherein said loading of said guide rod is done when said guide rod is non-coincidental to said longitudinal axis.
